(19) United States
(12) Patent Application Publication
(10) Pub. No.: US 2003/0124153 A1
(43) Pub. Date: Jul. 3, 2003

(54) Cosmetic Stick Compositions With Improved Application

(75) Inventors: Janet Elizabeth Opel, Brick, NJ (US); Renee Joan Ordino, Edison, NJ (US); Robert Walter Sandewicz, Spotswood, NJ (US)

(73) Assignee: Revlon Consumer Products Corporation, New York, 10022

(21) Appl. No.: 10/065,969

(22) Filed: Dec. 5, 2002

Related U.S. Application Data
(63) Continuation of application No. 09/679,217, filed on Oct. 25, 2000.
Application No. 09/679,217 is a continuation of application No. 09/086,462, filed on May 29, 1998, now abandoned.
Application No. 09/086,462 is a continuation-in-part of application No. 08/918,134, filed on Aug. 27, 1997, now Pat. No. 6,080,390.
Application No. 09/086,462 is a continuation-in-part of application No. 08/980,341, filed on Nov. 28, 1997, now Pat. No. 6,086,859.

(51) Int. Cl. A61K 7/00

(52) U.S. Cl. 424/401

(57) ABSTRACT

### Abstract of Disclosure

A cosmetic stick compositions comprising 0.5-30% of a stick structuring agent which is a solid or semi-solid at room temperature and has a melting point somewhere in the range of about 34 to 38^{o} C., 10-90% of a nonvolatile oil which is a guerbet ester, 1-30% of a wax comprising a fatty alcohol, and 0.1-25% of particulate matter having a particle size of 0.5 to 200 microns

### Cross Reference to Related Applications

This application is a continuation of U.S. patent application serial no. 09/679,217, filed October 25, 2000, which is a continuation of U.S. patent application serial no. 09/086,462, filed May 29, 1998, which is a continuation-in-part of U.S. patent application serial no. 08/918,134, filed August 27, 1997, and U.S. patent application serial no. 08/980,341, filed November 28, 1997, all of which are hereby incorporated by reference in their entirety

### Background of Invention

Technical Field

The invention is the field of cosmetic stick compositions, particularly pigmented cosmetic stick composition for application to lips, and a method for improving application characteristics thereof.

Cosmetic stick compositions which are applied to skin or lips must have application characteristics which are consumer acceptable. For example, if a cosmetic stick such as a lipstick provides excellent color and adherence to skin, yet is difficult to apply to the lips, it will not be a successful commercial product. Thus, application characteristics must be very carefully manipulated to ensure that a cosmetic stick applies well, feels good on the skin, and at the same time provides the cosmetic effect it is being applied for. One important application characteristic of cosmetic sticks, particularly lipstick is referred to as glide or slip. Glide refers to how well the cosmetic stick composition slips across the skin as it is applied. If one were to apply a crayon to the lips, even with relatively light pressure, the glide would be very poor, i.e. the crayon would drag across the lips with a frictional force which actually pulls and stretches the skin of the lips as the stick is moved across the lips. On the other hand, lipsticks which exhibit adequate glide will slide smoothly across the lips without pulling or stretching the skin, while being pressed against the lips with enough pressure to cause payoff, i.e. the laying down of a coating on the skin. It is important to regulate glide very carefully because, while sometimes a stick may glide easily across the lips, if sticks exhibit excessive glide they are very difficult to control, and feathering and bleeding of the applied stick into lines around the lip more readily occurs. In general, high viscosity oils contribute to glide. However, when lipsticks are formulated with these oily ingredients to maximize glide, the lipsticks may tend to be too oily. Thus, it is advantageous to use a glide enhancing agent which has a dual function, i.e. it contributes to stick structure while the stick is at room temperature, but when the stick is contacted to the skin or lips, the surface temperature of the skin or lips will cause the glide enhancing to melt or liquify and form an oil, which enhances glide. For example, U.S. Patent No. 4,996,044 teaches moisturizing lipstick compositions which may contain ingredients such as mineral oil and castor oil which the patentee states will improve glide. However lipsticks with such appreciable concentrations of such oils as glide agents tend to have a heavy, oily feel on the lips that most wearers dislike, and are more prone to feathering and bleeding into surrounding lip lines. In addition, the oily glide agents cannot help the stick to hold its shape at room temperature.

U.S. Patent No. 5,707,612 teaches castor oil/polyurethane derivatives for use in lipsticks. These polymers are liquid at room temperature and cannot act as stick structuring agents when the stick is maintained at room temperature.

Accordingly there is a need for a lipstick which exhibits improved glide without a heavy, oily feel or an unacceptable tendency toward feathering or bleeding, and which holds its shape well at room temperature.

Another object of the invention is to provide a cosmetic stick composition where the glide agent is not an oil when the stick is maintained at room temperature, but transforms into an oil when contacted with the skin. In this way, formulating sticks with appreciable levels of heavy oils can be avoided.

The object of the invention is to provide a cosmetic stick composition which exhibits improved glide, but without negatively impacting control of the stick when it is applied to the lips.

Another object of the invention is to provide a cosmetic composition which moisturizes the lips.

### Summary of Invention

The invention comprises a method for improving the glide of cosmetic stick composition comprising formulating the stick composition with an effective amount of a stick structuring agent which is a solid or semi-solid at room temperature, i.e. 25^{o} C., and has a melting point somewhere in the range of about 34-38^{o} C., whereby the stick structuring agent present in the composition melts upon exposure to skin or lips of average surface temperature during application, causing the stick to glide more easily across the skin.

The invention also comprises a cosmetic stick composition comprising, by weight of the total composition:

0.5-30% of a stick structuring agent which is a solid or semi-solid at room temperature and has a melting point somewhere in the range of about 34 to 38^{o} C.

10-90% of a nonvolatile oil which is a guerbet ester which is a liquid at room temperature and has a viscosity of about 50 to 500 centipoise at 25^{o} C.,

1-30% of a wax having a melting point of about 39 to 135^{o} C., and

0.1-25% of particulate matter having a particle size of 0.5 to 200 microns.

### Detailed Description

All percentages mentioned herein are percentages by weight unless otherwise indicated.

The term The term stick refers to cosmetic compositions having a consistency such that they can be molded into the form of a stick -- for instance by being heated until molten and then poured into a mold and cooled. Also included within the definition of stick are compositions of the invention that are capable of being formed into sticks, but are poured into pans or other types of cake or cream forms to deliver certain consumer benefits. For example, an eyeshadow composition in accordance with the invention may be molded in the stick form, but it may be desired to pour it into a pan because this container is more desireable from a consumer standpoint.

THE METHOD OF THE INVENTION

In the method of the invention, the application characteristics, including glide or slip, of a cosmetic stick composition is improved by adding to the composition an effective amount of a stick structuring agent which is a solid or semi-solid at room temperature and which has a melting point somewhere in the range of about 34 to 38^{o} C., preferably 34 to 36^{o} C., most preferably 35 to 36^{o} C. The temperature at which the stick structuring agent melts most closely approximates the surface temperature of skin, which is slightly less than body temperature. The term melt when used in accordance with the invention, refers to the transition of the stick structuring agent from a solid or semi-sold to a state where the structuring agent is capable of spreading on the skin, in other words a liquid or liquid-like state. The stick structuring agent stabilizes or assists in stabilizing the stick structure when the stick is maintained at room temperature. However, when the cosmetic stick is gently drawn across the skin, the contact of the skin (having a surface temperature of about 35^{o} C.) with the stick will cause the structuring agent in the stick to melt or liquify, thereby creating an oily film that helps the stick glide across the skin or lip surface as it is applied. The amount of stick structuring agent necessary to improve glide is generally somewhere in the range of about 0.5 to 30%, preferably 1.5-25%, more preferably 3-15% by weight of the total composition. Preferably, the compositions are substantially anhydrous.

THE COMPOSITIONS OF THE INVENTION

The Stick Structuring Agent

The cosmetic stick compositions of the invention comprise a stick structuring agent which is a solid or semi-solid at room temperature and has a melting point somewhere in the range of about 34 to 38^{o} C., preferably in the ranges specified above. To function as a stick structuring agent the material is preferably an oleophilic organic compound, or a mixture of such. Also, it may be preferred that it contain some hydrophilic groups too, to help hold the wax and particulate ingredients in intimate admixture with one another. A variety of stick structuring agents exhibit these characteristics. For example, lanolin may be suitable for use in the sticks of the invention. Preferred are stick structuring agents which are non-crystalline organic compounds and have moderate to extensive branching in their chemical structures. Certain sterols are particularly suitable. Sterols are animal or vegetable derived isocyclic compounds which exhibit a tetracyclic cyclopenteneophenanthrene skeleton as set forth below:

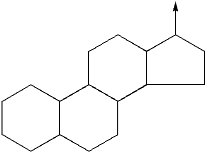

The above skeleton may contain hydroxyl or keto groups, some ring unsaturation, methyl groups, or aliphatic side chains at the A, or C-17, position. The hydroxyl groups may be esterified with fatty acids. Preferred are sterols which are esterified with branched chain fatty acids, in particular, C10-30 cholesterol/lanasterol esters, which are formed by the esterification of cholesterol and lanasterol with a mixture of C10-30 fatty acids wherein the fatty acids are a mixture of straight or branched chain fatty acids, or are all branched chain fatty acids. Particularly preferred is noncrystalline mixture of C10-30 cholesterol/lanesterol esters having a melting point in the range of about 30 to 38^{o} C., which is sold by Croda Oleochemicals under the tradename Super Sterol ester, a highly diverse complex derived from wool wax, characterized by a combination of branched chain fatty acids, cholesterol and related sterols. Its fatty acid component is a combination of normal, iso, and anteiso fatty acids.

The Nonvolatile Oil

The composition of the invention contains 10-90%, preferably 15-75%, more preferably 20-60% by weight of the total composition of a nonvolatile oil which is a liquid at room temperature and has a viscosity in the range of about 50 to 500, preferably 75-300, most preferably 100-200 centipoise at 25^{o} C. The term nonvolatile means that the oil has a vapor pressure of less than about 2 mm. of mercury at 20^{o} C.

Examples of nonvolatile oils include guerbet esters, which are generally defined as esters which are formed by the reaction of a guerbet alcohol (which is a branched chain alcohol) having the general formula:

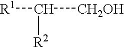

with a carboxylic acid having the general formula:

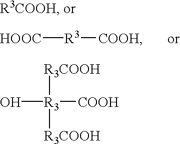

wherein R¹ and R² are each independently a C₄₋₂₀ alkyl and each R³ is a substituted or unsubstituted C₁₋₅₀ straight or branched chain alkyl or alkylene group, or phenyl, wherein the substituents are halogen, hydroxyl, carboxyl, or alkylcarbonyloxy. Preferred are guerbet esters which are the reaction product of a guerbet alcohol as identified above, and a compound of the formula:

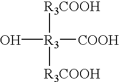

wherein each R₃ is independently a C₁₋₁₀ straight or branched alkyl or alkylene. Preferably each R₃ is C₁ alkyl or alkylene, e.g. the compound is citric acid (2-hydroxy-1,2,3-propanetricarboxylic acid). Examples of such guerbet esters are those disclosed in U.S. Patent No. 4,868,236, which is hereby incorporated by reference. Particularly preferred is where the nonvolatile oil is trioctyldodecyl citrate, which is sold by Phoenix Chemical Inc. under the tradename Pelemol TGC. Trioctyl dodecyl citrate has the following general formula:

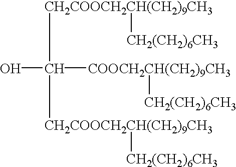

Other examples of nonvolatile oils suitable for use in the cosmetic sticks of the invention include esters of the formula RCO-OR' wherein R and R' are each independently a C₁₋₂₅, preferably a C₄₋₂₀, straight or branched chain alkyl, alkenyl or alkoxycarbonylalkyl or alkylcarbonyloxyalkyl. Preferred are esters which are the reaction product of of a branched chain fatty acid and a branched or straight chain fatty alcohol, preferably a branched chain fatty alcohol. Examples of such esters include isotridecyl isononanoate, isostearyl neopentanoate, tridecyl neopentanoate, cetyl octanoate, cetyl ricinoleate, decyl isostearate, isodecyl oleate, isodecyl neopentanoate, isohexyl neopentanoate, tridecyl octanoate, and so on. Preferably, the nonvolatile oil component of the compositions contains from about 10-100% by weight of the total nonvolatile oil component of esters which are the reaction products of either branched chain fatty acids or branched chain alcohols, such that either the fatty acid moiety or the fatty alcohol moiety contains some branching.

The oil may also comprise naturally occuring glyceryl esters of fatty acids, or triglycerides. Both vegetable and animal sources may be used. Examples of such oils include castor oil, lanolin oil, triisocetyl citrate, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable as the oil are synthetic or semi-synthetic glyceryl esters, e.g. fatty acid mono-, di-, and triglycerides which are natural fats or oils that have been modified, for example, acetylated castor oil, glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates, PEG glyceryl tallowates, and so on.

Also suitable as the oil are nonvolatile hydrocarbons such as isoparaffins, hydrogenated polyisobutene, mineral oil, squalene, petrolatum, and so on.

Nonvolatile silicones, both water soluble and water insoluble, are also suitable as the oil component. Suitable water insoluble nonvolatile silicones include amodimethicone, dimethicone, phenyl trimethicone, and mixtures thereof. Also suitable are water soluble silicones such as dimethicone copolyol, dimethiconol, and the like. Such silicones are available from Dow Corning as the 3225C formulation aid, Dow 190 and 193 fluids, or similar products marketed by Goldschmidt under the ABIL tradename.

Also suitable as the nonvolatile oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. patent no. 5,118,496 which is hereby incorporated by reference. Perfluoropolyethers like those disclosed in U.S. patent nos. 5,183,589, 4,803,067, 5,183,588 all of which are hereby incorporated by reference, which are commercially available from Montefluos under the trademark Fomblin, and which tend to enhance the shine of the lipstick, for use as the nonvolatile oil component.

The preferred cosmetic stick compositions of the invention contain a mixture of a guerbet ester of citric acid and a monoester of a branched chain fatty acid and a branched chain fatty alcohol. More preferably the nonvolatile oil is a mixture of trioctyldodecyl citrate and isotridecyl isononanoate in a range of 25-65% by weight of the total composition of the guerbet ester and 15-35% by weight of the total composition of the ester which is the reaction product of a branched chain fatty acid and a branched chain alcohol. Most preferred is where the cosmetic stick composition is free of volatile solvents or contains volatile solvents in very minimal amounts, preferably less than 5% by weight, more preferably less than 1% by weight. The term volatile solvent means a solvent which has a vapor pressure of at least 2 mm. of mercury at 20^{o} C. Examples of volatile solvents include volatile linear and cyclic silicones, or paraffinic hydrocarbons, short chain alcohols such as ethanol and isopropanol, and the like.

The Wax

The cosmetic stick compositions of the invention contain 1-30%, preferably 1-25%, more preferably 3-20% by weight of the total composition of a wax having a melting point of about 39 to 135^{o} C., preferably in the range of 45 to 95^{o} C., most preferably 55 to 95^{o} C. Suitable waxes generally includes animal waxes, plant waxes, mineral waxes, silicone waxes, synthetic waxes, and petroleum waxes. Examples of waxes in accordance with the invention include bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, stearyl dimethicone, and the like, as well synthetic homo- and copolymer waxes such as PVP/eicosene copolymer, PVP/hexadecene copolymer, and the like. Preferably the wax component of the cosmetic stick will contain a mixture of synthetic waxes and natural waxes in a range of about 5-20%, preferably 7-15% synthetic wax and about 0.5-10%, preferably 1-7% animal or vegetable wax.

Particularly preferred is where the synthetic wax is an ethylene homopolymer or ethylene copolymer. The molecular weight of the ethylene homopolymer and/or copolymers used as the wax component may vary, so long as the melting point of the homo- or copolymer either alone or in combination is not greater than 135^{o} C. Generally polyethylene waxes having a melting point range of 30 to 135^{o} C. will have a molecular weight ranging from about 100 to about 2,000. Preferably the ethylene copolymers are comprised of ethylene monomer units in either repetitive or randon sequence, in combination with monomer units derived from an ethylenically unsaturated comonomer of the following formula:

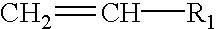

wherein R₁ is a C₁₋₃₀ straight or branched chain saturated or unsaturated alkyl, aryl, or aralkyl, preferably a C₁₋₁₀ straight or branched chain alkyl. Examples of ethylene homo- and copolymers which may be used in the invention are set forth in U.S. Patent No. 5,556,613, which is hereby incorporated by reference.

Particulate Matter

The compositions of the invention comprise 0.1-25%, preferably 0.5-20%, more preferably 1-18% by weight of the total composition of particulate matter having a particle size of 0.5 to 200, preferably 1-100 microns. The particulate matter may be colored or non-colored (for example white) non-pigmentitious powders that may give the cosmetic stick an opaque or semi-opaque quality and contribute to stick structure. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller"s earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in cosmetic sticks it is used more for its ability to mute color, and/or provide an opaque or semi-opaque finish, then as a colorizing ingredient. The above mentioned powders may be surface treated with lecithin, amino acids, mineral oil, silicone oil or various other agents either alone or in combination, which coat the powder surface and render the particles more lipophilic in nature.

The particulate matter component also may comprise various organic and/or inorganic pigments, alone or in admixture with one or more non-pigmentatious powders. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmentatious and non-pigmentatious particulate matter. The percentage of pigment used in the particulate matter component will depend on the type of cosmetic being formulated.

Other Ingredients

The compositions of the invention may contain other ingredients which maximize the beneficial moisturizing effects of the composition as set forth below.

Hydrophobic Agents

The composition of the invention may contain one or more hydrophobic agents which enhance glide. Suggested ranges of hydrophobic agents are 0.01-10%, preferably 0.05-8%, more preferably 0.1-5% by weight of the total composition. Suitable hydrophobic agents are hydrophobic clays and/or hydrophobic silicas. Examples of hydrophobic clays are hydrophobically treated hectorite and bentonite clays, such as those sold under the tradenames Bentone 27 and 38 by Rheox Corporation. The hectorite and bentonite clay minerals are generally described as three layer clays where a sheet of aluminum/oxygen atoms or magnesium/oxygen atoms lies between two layers of silicon/oxygen atoms, i.e. aluminosilicates and magnesium silicates. The clays may be reacted with cationic surfactants such as quaternary ammonium cationic surfactants. Particularly preferred for use in the cosmetic sticks of the invention are clays reacted with cationic surfactants such as ditallow dimethyl ammonium chloride, which is known by the CTFA name Quaternium-18 hectorite.

Also suitable as the hydrophobic agent are hydrophobic silicas which are generally formed by chemically modifying the silanol groups on the silica surface with halosilanes, alkoxysilanes, or siloxanes. Suitable hydrophobic silicas are available from Degussa Corporation under the tradename Aerosil, or Cab-O-Sil Corporation under the tradnems TS530, TS610, TS720 and the like.

Preservatives

The composition may contain 0.0001-8%, preferably 0.001-6%, more preferably 0.005-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, propyl paraben, methyl paraben, benzoic acid, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D, C, and K, as well as derivatives thereof are suitable. Particularly preferred are derivatives of vitamins C, E, and A such as magnesium ascorbyl phosphate, retinyl palmitate, tocopheryl acetate, and mixtures thereof.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

Biological Additives

It may also be desireable to include 0.0001-5%, preferably 0.0005-4%, more preferably 0.001-3% by weight of one or more biological additives that provide a subtle conditioning effect to the skin. Biological additives are materials which are generally derived from plants, and in some cases animals. Examples of suitable biological additives include aloe, sodium hyaluronate, plant extracts, plant derived oils and the like. Particularly preferred are aloe and sodium hyaluronate.

Preferred is a composition containing one or more of the above ingredients either alone or in combination. Preferably, the ingredients are pre-formed as a complex, and added to the lipstick composition.

The invention will be further described in connection with the following examples which are set forth for the purposes of illustration only.

EXAMPLE 1

A red lipstick was made according to the following formula:

The lipstick was made by grinding the pigments in a portion of the trioctyldodecyl citrate and quaternium 18 hectorite. The oils were combined and the pigments were added to the oils. The melted waxes were added to the oil/pigment mixture with stirring. The lipstick composition was poured into molds and allowed to cool.

EXAMPLE 2

Panelists who were experts in lipstick applied the lipstick composition of Example 1 to their lips and rated glide according to the following scale:

EXAMPLE 3

The lipstick of Example 1 (hereinafter Formula I) was compared with a lipstick having the following formula (hereinafter Formula II):

Five panelists applied the lipstick of Formula II to the lips and rated the application according to the scale set forth in Example 2. After cleaning their lips with Almay oil-free eye makeup remover pads, the same panelists applied the lipstick of Formula I to their lips and rated the application according to the scale set forth in Example 2. All five panelists rated the lipstick of Formula I a 4 on the 0 to 4 scale, i.e. the lipstick of Formula I was easy to apply, had no drag, and did not pull the lips. All five panelists rated the lipstick of Formula II, a 2 on the 0 to 4 scale, i.e. the lipstick was moderately difficult to apply, some drag was evident, as well as a slight pull on the lips. Thus, the lipstick of the invention has improved application.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

Claims

1. A cosmetic stick composition comprising, by weight of the total composition:
0.5-30% of a stick structuring agent which is a solid or semi-solid at room temperature and has a melting point of 34 to 38^{o} C.
10-90% of a nonvolatile oil which is selected from the group consisting triisostearyl citrate, trioctyldodecyl citrate, and mixtures thereof;
1-30% of a wax comprising a fatty alcohol, and
0.1-25% of particulate matter having a particle size of 0.5 to 200 microns.

2. The composition of claim 1 wherein the stick structuring agent is an oleophilic organic compound.

3. The composition of claim 2 wherein the stick structuring agent further comprises hydrophilic groups.

4. The composition of claim 1 wherein the stick structuring agent comprises lanolin.

5. The composition of claim 1 wherein the stick structuring agent is non-crystalline organic compound having moderate to extensive branching in its chemical structure.

6. The composition of claim 1 wherein the stick structuring agent is a sterol or sterol ester.

7. The composition of claim 1 wherein the sterol has the formula: 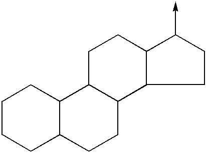
wherein A is methyl or an aliphatic side chain.

8. The composition of claim 1 wherein the stick structuring agent is a sterol that contains hydroxyl groups esterified with one or more fatty acids.

9. The composition of claim 8 wherein the fatty acids are branched chain fatty acids.

10. The composition of claim 1 wherein the stick structuring agent comprises a sterol esterified with branched chain fatty acids.

11. The composition of claim 1 wherein the stick structuring agent comprises C10-30 cholesterol/lanasterol esters.

12. The composition of claim 1 wherein the fatty alcohol comprises cetyl alcohol.

13. The composition of claim 1 further comprising cyclomethicone

14. The composition of claim 1 further comprising polyethylene wax.

15. A cosmetic stick composition comprising, by weight of the total composition:0.5-30% of a stick structuring agent which is a solid or semi-solid at room temperature and has a melting point of 34 to 38^{o} C.10-90% of a nonvolatile oil which is the reaction product of a guerbet alcohol of the formula: 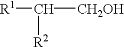
with a carboxylic acid having the general formula: 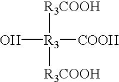
wherein R¹ and R² are each independently a C₄₋₂₀ alkyl and each R³ is a C₁₋₅₀ straight or branched chain alkyl.
1-30% of a wax comprising a fatty alcohol, and
0.1-25% of particulate matter having a particle size of 0.5 to 200 microns.

16. The composition of claim 15 wherein R₁ is methyl and the carboxylic acid is citric acid.

17. The composition of claim 16 wherein the guerbet alcohol is octyldodecyl alcohol, isostearyl alcohol.

18. The composition of claim 17 wherein the wax comprises cetyl alcohol.

19. The composition of claim 16 further comprising acrylates copolymer.

20. A lipstick composition comprising, by weight of the total composition:
0.5-30% of a stick structuring agent which is a solid or semi-solid at room temperature and has a melting point of 34 to 38^{o} C.
10-90% of a non-volatile oil comprising triisostearyl citrate,
1-30% of a wax comprising a fatty alcohol,
0.1-25% of particulate matter having a particle size of 0.5 to 200 microns.
